(19) United States
(12) Patent Application Publication
(10) Pub. No.: US 2019/0374286 A1
(43) Pub. Date: Dec. 12, 2019

(54) LASER IRRADIATION DEVICE

(71) Applicant: Pukyong National University Industry-University Cooperation Foundation, Busan (KR)

(72) Inventor: Hyun Wook Kang, Busan (KR)

(73) Assignee: Pukyong National University Industry-University Cooperation Foundation, Busan (KR)

(21) Appl. No.: 16/184,155

(22) Filed: Nov. 8, 2018

(30) Foreign Application Priority Data
Jun. 8, 2018 (KR) 10-2018-0066146

(51) Int. Cl. A61B 18/20; H01S 3/11

(52) U.S. Cl. CPC A61B 18/20; A61B 2018/00589; H01S 3/11

(57) ABSTRACT

A laser irradiation device for irradiating laser to a tissue includes: a decrease form determination unit configured to determine a decrease form of laser power according to a laser irradiation time; a decrease rate determination unit configured to determine a decrease rate of the determined decrease form; a laser light source unit configured to emit laser in the determined decrease form and the determined decrease rate; and a laser irradiation unit configured to receive laser emitted from the laser light source unit and irradiate the received laser. A constant photoreaction can be induced by constantly maintaining a temperature in a site to be irradiated with laser.

### CROSS-REFERENCE TO RELATED APPLICATIONS

This application claims the benefit of Korean Patent Application No. 10-2018-0066146, filed Jun. 8, 2018, in the Korean Intellectual Property Office. The entire contents of said application is incorporated herein by reference for all purposes.

### BACKGROUND OF THE INVENTION

### Field of the Invention

The present invention relates to a laser irradiation device, and more particularly, to a laser irradiation device which is usable for various surgeries and therapies.

### Description of the Related Art

Laser energy is used in various surgical and therapeutic procedures for clinical applications. Such surgical and therapeutic procedures include incision, extraction, removal, vaporization, resection, destruction, coagulation, hemostasis, and curing, and are currently applied to various parts of the human body.

In a conventional therapeutic procedure, for example, a laser having a wavelength of 532 nm, which is well absorbed in blood vessels present in tissues, has been used. As another example, laser interstitial thermal treatment (LITT) is used for the treatment of cancer in tissues. In this case, infrared wavelengths (808 nm, 980 nm, 1064 nm, or the like) are used.

However, when a laser wavelength used for therapy is irradiated with constant laser power as in a conventional method, a temperature difference due to a thermal gradient or a space greatly occurs as it goes down from the tissue surface to the deep part. In particular, in order to induce photocoagulation in the deep tissue, laser energy has to be irradiated for a long time or laser power has to be increased. In this case, carbonization occurs on the tissue surface due to an excessive temperature increase, which adversely affects the therapeutic effect.

In order to minimize a temperature change in tissues and prevent carbonization from occurring on the tissue surface, it is necessary to induce predictable photoreaction (photocoagulation) while keeping the temperature in the tissues constant.

In order to solve the problems, it is essential to know parameters that have a major influence on the tissue reaction among various parameters of a laser system that affect the temperature change in the tissues, and furthermore, to know a correlation between the variation of the parameters and the tissue reaction.

### SUMMARY OF THE INVENTION

One or more embodiments of the present invention provide a laser irradiation device that reduces laser power with time and irradiates laser.

According to one or more embodiments of the present invention, a laser irradiation device for irradiating laser to a tissue includes: a decrease form determination unit configured to determine a decrease form of laser power according to a laser irradiation time; a decrease rate determination unit configured to determine a decrease rate of the determined decrease form; a laser light source unit configured to emit laser in the determined decrease form and the determined decrease rate; and a laser irradiation unit configured to receive laser emitted from the laser light source unit and irradiate the received laser.

The decrease form may be determined in consideration of parameters of at least one of a photocoagulation duration time and a coagulation range in a tissue.

The decrease form may be a linear decrease form.

The decrease form may be an exponential decrease form.

The decrease rate may be determined in consideration of parameters of at least one of an irradiation time, a coagulation thickness in a tissue, and a tissue temperature for coagulation induction.

When the laser is in a continuous wave form, a temperature in the tissue may be maintained at a specific temperature for a predetermined time, upon laser irradiation through the laser irradiation device.

When the laser is in a pulsed or quasi-continuous pulsed form, an average value of temperatures in the tissue may be maintained at a specific value for a predetermined time, upon laser irradiation through the laser irradiation device.

The effects of the laser irradiation device according to embodiments of the present invention are as follows:

The constant photoreaction can be induced by constantly maintaining a temperature in a site to be irradiated with laser.

In addition, when laser is irradiated while decreasing laser power with time, no carbonization occurs on the tissue surface, and laser energy absorption and heat transfer are very efficiently performed, so that the coagulation range in the tissue can be maximized.

In addition, since the linear or exponential decrease form can be selected according to the coagulation range in the tissue, effective therapy is possible.

However, the effects that the laser irradiation device according to embodiments of the present invention can achieve are not limited to those mentioned above, and other advantages not mentioned will be obviously understood by those skilled in the art from the following description.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram illustrating an overall configuration of a laser irradiation device 1000 according to an embodiment of the present invention;

FIG. 2 is a block diagram illustrating a control unit 110 according to an embodiment of the present invention;

FIGS. 3A and 3B are graphs showing a change in laser power when a laser wavelength is in a continuous form, upon laser irradiation through the laser irradiation device 1000 according to an embodiment of the present invention;

FIGS. 4A and 4B are graphs showing a change in laser power when a laser wavelength is in a pulsed form, upon laser irradiation through the laser irradiation device 1000 according to an embodiment of the present invention;

FIGS. 5A and 5B are graphs showing a change in laser power when a laser wavelength is in a quasi-continuous pulsed form, upon laser irradiation through the laser irradiation device 1000 according to an embodiment of the present invention;

FIGS. 6A and 6B are graphs showing a temperature change in a tissue when a laser wavelength is in a continuous form, upon laser irradiation through the laser irradiation device 1000 according to an embodiment of the present invention;

FIGS. 7A and 7B are graphs showing a temperature change in a tissue when a laser wavelength is in a pulsed form, upon laser irradiation through the laser irradiation device 1000 according to an embodiment of the present invention;

FIGS. 8A and 8B are graphs showing a temperature change in a tissue when a laser wavelength is in a quasi-continuous pulsed form, upon laser irradiation through the laser irradiation device 1000 according to an embodiment of the present invention;

FIGS. 9A and 9B show comparison of a photocoagulation reaction between a case in which laser is irradiated with constant laser power and a case in which laser is irradiated while reducing laser power with time according to the present invention; and

FIG. 10 shows an error of a coagulation volume according to a decrease form, upon laser irradiation through the laser irradiation device 1000 according to an embodiment of the present invention.

### DESCRIPTION OF THE PREFERRED EMBODIMENTS

Hereinafter, exemplary embodiments of the present invention will be described in detail with reference to the accompanying drawings.

Also, while describing the present invention, detailed descriptions about related well-known functions or configurations that may diminish the clarity of the points of the present invention are omitted.

The terms and words used in the present specification and claims should not be construed as limited to ordinary or dictionary meanings, and should be interpreted as the meaning and concept corresponding to the technical idea of the present invention based on the principle that the inventors can appropriately define the concept of the terms in order to explain its own invention in the best way. Therefore, the embodiments described in the present specification and the configurations shown in the drawings are only the most preferred embodiments of the present invention, and do not represent all of the technical ideas of the present invention. It should be understood that various equivalents and modifications may be substituted for those at the time of filing of the present application.

Hereinafter, a laser irradiation device according to an embodiment of the present invention will be described in detail with reference to the accompanying drawings

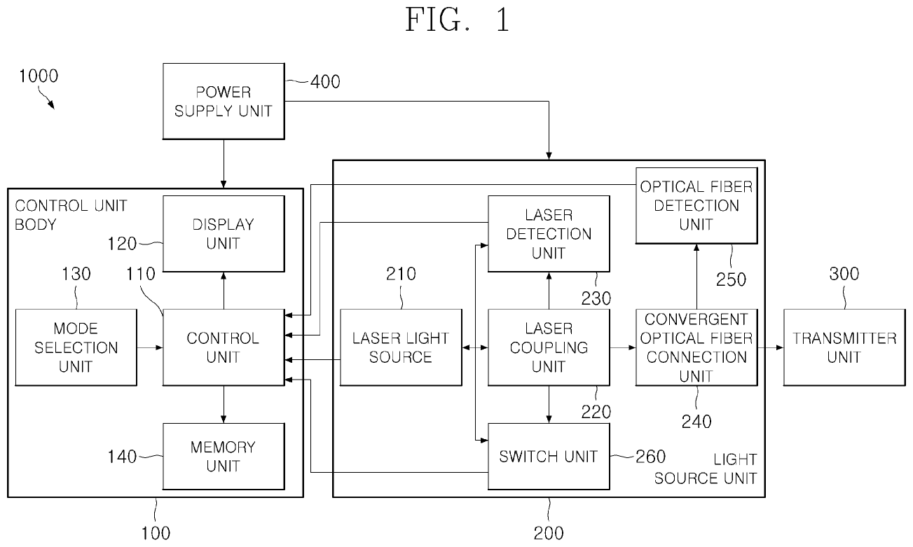

FIG. 1 is a block diagram illustrating an overall configuration of a laser irradiation device 1000 according to an embodiment of the present invention.

The laser irradiation device 1000 according to the embodiment of the present invention reduces laser power with time, without irradiating constant laser power, so as to minimize a temperature change in tissues and prevent carbonization on the tissue surface, thereby inducing a predictable photoreaction.

The laser irradiation device 1000 according to the embodiment of the present invention may include a control unit body 100, a light source unit 200, and a transmitter unit 300.

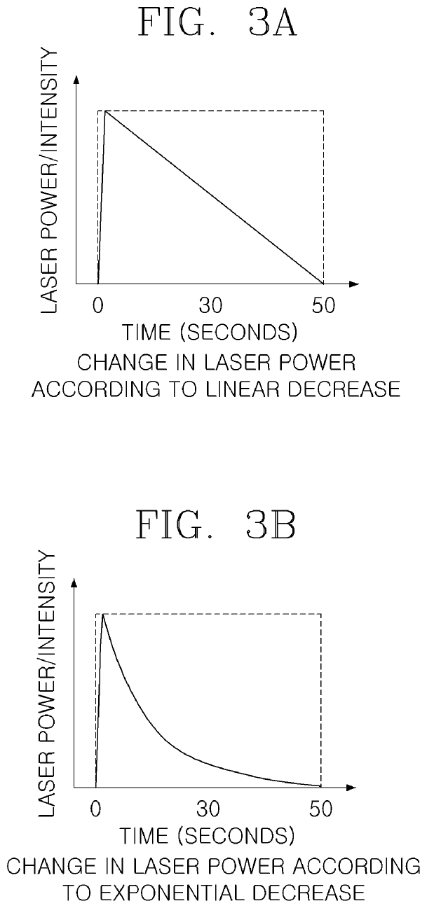

The control unit body 100 includes a control unit 110, a display unit 120, a mode selection unit 130, and a memory unit 140. The control unit 110 includes a laser power control unit 112 that directly controls laser power, a pulse length control unit 114 that controls a pulse length according to laser power, and a repetition rate control unit 116 that controls a pulse interval for each laser energy or laser power. The control unit 110 emits laser having power, energy, irradiation time, pulse length, and repetition rate, which are designated according to a selection mode inputted from the mode selection unit 130. The display unit 120 displays a mode that is selectable by a user, and displays an operation state. The mode selection unit 130 includes all selection buttons and receives a desired driving mode from the user. The memory unit 140 stores the power, energy, irradiation time, pulse length, and repetition rate for each mode. The control unit 110 will be described below in detail with reference to FIG. 3.

The light source unit 200 includes a laser light source 210 that generates a laser wavelength in a continuous form, a pulsed form, or a quasi-continuous pulsed form, a laser coupling unit 220 that couples laser energy, a laser detection unit 230 that automatically controls laser power decreasing with time, a switch 260 that confirms optical coupling and checking a laser safety state, a convergent optical fiber connection unit 240 that integrates electromagnetic energy generated by the transmitter into the optical fiber, and an optical fiber detection unit 250 that checks an optical fiber connection state and notifies the display unit of the checked optical fiber connection state.

A diode laser, a flash-lamped laser, a frequency multiplied solid state laser, an ultraviolet/IR flash lamp, a light emitting diode (LED), an infrared bulb, or a DPSS may be used as the laser light source 210.

A continuous wave laser light source may use a light source that includes a diode and a driver. A gain medium that is usable in a pulsed wave or quasi-continuous wave laser light source may be Ho:YAG, Tm:YAG, Tm:Ho:YAG, Er:YAG, Er:YLF, Er:YSGG, Nd:YAG, Tm-fiber laser, or CTH:YAG.

In addition, a pumping light source for generating a quasi-continuous wave may generate high power light energy through pumping of a diode pumped-solid state (DPSS) laser and a resonator. The pumping DPSS laser used for generating the quasi-continuous wave may have a wavelength of 800 nm to 1,040 nm. The pumping DPSS laser may receive a signal for a selection mode from the control unit, drive the pumping light source according to the power, pulse length, and pulse repetition rate, and generate a desired wavelength, laser energy, and irradiation mode through the resonator.

Laser energy generated by the laser light source 210 according to the mode selection may be integrated into the optical fiber through the laser coupling unit 220. The laser detection unit 230 may check the integrated laser energy, and the optical fiber detection unit 250 may automatically check the optical fiber connection and the laser energy integration.

Meanwhile, the laser irradiation device 1000 according to the embodiment of the present invention includes the transmitter unit 300 that may transmit the generated electromagnetic energy to an endoscope or a narrow channel, and a power supply unit 400 that supplies power to the control unit body 100 and the light source unit 200.

The laser irradiation device 1000 according to the embodiment of the present invention irradiates laser by presetting the power, pulse length, and repetition rate before laser irradiation, and constantly maintain the temperature in the tissue during tissue therapy, thereby inducing effective photocoagulation. During the tissue therapy, the tissue temperature setting of the tissue is possible through the mode selection unit 130.

In consideration of optical characteristics, the temperature preferable for inducing the photocoagulation of the tissue is 60° C. to 70° C. For example, when the wavelength of the laser used is 532 nm, at which vascular absorption in the tissue is strongly exhibited, the absorption coefficient (μa) of the tissue is 100 cm⁻¹, and when the energy density is 1,000 J/cm², it can be predicted using Equation 1 that the temperature increase is 23° C.

ΔT=μa×H/ρ·c (Equation 1)

(ΔT=temperature variation (K), H=energy density (J/cm²), ρ=material density (kg/m³), c=specific heat of static pressure (J/kg·K)).

After setting the temperature for laser coagulation induction, the coagulation depth in the tissue may be set according to coagulation. The coagulation depth may be set to 0.3 mm to 3 mm according to the tissue characteristics and recovery, and the pulse length and the repetition rate may be set according to the coagulation depth.

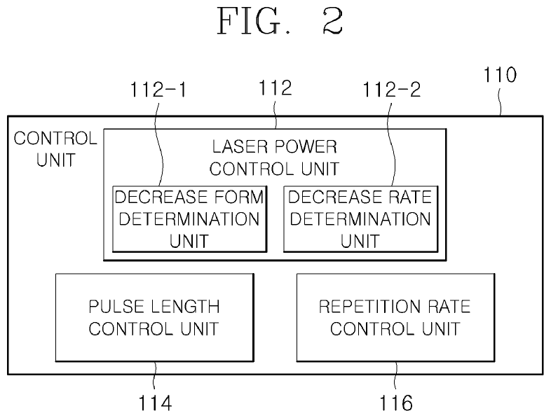

FIG. 2 is a block diagram illustrating the control unit 110 according to an embodiment of the present invention.

The control unit 110 according to the embodiment of the present invention includes the laser power control unit 112 that directly controls the laser power, the pulse length control unit 114 that controls the pulse length according to the laser power, and the repetition rate control unit 116 that controls the pulse interval for each laser energy or laser power. The control unit 110 may irradiate laser by presetting the power, pulse length, and repetition rate before laser irradiation.

In the case of the continuous wave laser, the range of the laser power controlled by the laser power control unit 112 may be 0.5 W to 1,000 W by controlling on/off of the laser power through modulation. In the case of the pulsed laser, the laser power of 100 W to 100 kW may be obtained according to the pumping method of the gain medium in the resonator. In the case of the quasi-continuous wave laser, the range of the laser power may be 100 W to 100 kW according to a pumping laser such as DPSS and a Q-switching control function. For example, when the diameter of the laser beam is 1 mm, the laser power density is 63.7 W/cm² to 127 kW/cm². In addition, when the pulse length is 500 μm, the energy density (fluence rate) may be 0.03 J/cm² to 63.7 J/cm².

In the case of the continuous wave laser, the range of the pulse length controlled by the pulse length control unit 114 may be 0.1 ms to 1,000 ms by electrically controlling on/off of the laser power through modulation. In the case of the pulsed laser, the pulse length of 0.5 μs to 300 μs may be obtained according to the pumping method of the gain medium in the resonator. In the case of the quasi-continuous wave laser, the range of the pulse length may be 10 ns to 250 ns according to a pumping laser such as DPSS and a Q-switching control function.

In the case of the continuous wave laser, the range of the repetition rate controlled by the repetition rate control unit 116 may be 1 Hz to 1 kHz by controlling on/off of the laser power through modulation. In the case of the pulsed laser, the repetition rate of 1 Hz to 100 Hz may be obtained according to the gain medium in the resonator and a repetition rate of a pumping device. In the case of the quasi-continuous wave laser, the range of the repetition rate may be 5 kHz to 35 kHz according to a pumping such as DPSS and a Q-switching frequency.

Meanwhile, the laser power control unit 112 includes a decrease form determination unit 112-1 that determines whether the decrease form of the laser power is a linear decrease form or an exponential decrease form, and a decrease rate determination unit 112-2 that determines a factor such as a slope in the case of the linear decrease and a magnitude of an exponent in the case of the exponential decrease.

When laser is irradiated through the laser irradiation device 1000 according to the embodiment of the present invention, a change in laser power when a laser wavelength is in a continuous form, a pulsed form, and a quasi-continuous pulsed form will be described with reference to FIGS. 3A to 5B.

FIGS. 3A and 3B are graphs showing a change in laser power when a laser wavelength is in a continuous form, upon laser irradiation through the laser irradiation device 1000 according to the embodiment of the present invention.

As illustrated in FIGS. 3A and 3B, the continuous wave laser power may be changed according to the irradiation time. The decrease form determination unit 112-1 of the laser power control unit 112 may select the decrease form of the laser power as a linear form or an exponential form. The laser power of the continuous wave laser may be set to 0.5 W to 1,000 W, the pulse length thereof may be set to 0.1 ms to 1,000 ms, and the repetition rate thereof may be set to 1 Hz to 1 kHz.

As compared with the general continuous wave laser that constantly generates laser power with time, the laser irradiation device 1000 according to the embodiment of the present invention irradiates laser power in a continuous decrease form according to the irradiation time.

The decrease form may decrease the laser power linearly according to the irradiation time or may decrease the laser power exponentially according to the irradiation time.

The decrease form may be determined in consideration of the photocoagulation temperature duration time or the coagulation range in the tissue. For example, when the photocoagulation duration time is within 35 seconds or the photocoagulation range is 2 mm or less, a linear function may be used, and when the photocoagulation duration time is 40 or more or the photocoagulation range is 2 mm or more, an exponential function may be used.

After the decrease form is determined, the decrease rate of the laser power may be determined according to the irradiation time, coagulation thickness in the tissue, and tissue temperature for coagulation induction. Upon linear decrease, when the slope decreases in the exponential form, the magnitude of the exponent may be adjusted.

In the case of the general continuous wave laser, the laser power is constantly generated, but in the case of the laser irradiation device 1000 according to the embodiment of the present invention, the intensity of the laser power decreases with time. Thus, the total energy amount transmitted to the tissue may be relatively small. Meanwhile, the laser irradiation device 1000 according to the embodiment of the present invention increases initial power so as to cause photocoagulation during laser irradiation, thereby keeping the total energy amount equal.

The laser power of the continuous wave laser may be adjusted in the on/off form by using the modulation method. This is similar to the pulsed wave laser. Upon continuous wave laser irradiation using the modulation method, the decrease form of the laser power may be applied in the linear or exponential form.

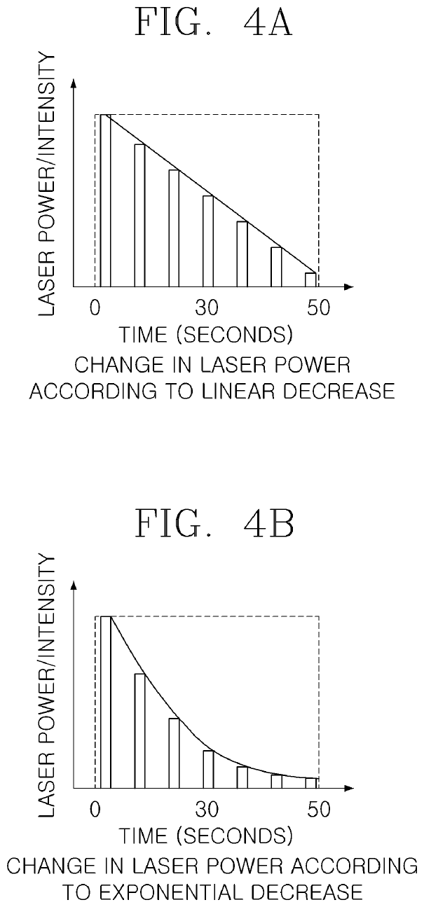

FIGS. 4A and 4B are graphs showing a change in laser power when a laser wavelength is in a pulsed form, upon laser irradiation through the laser irradiation device 1000 according to the embodiment of the present invention.

As illustrated in FIGS. 4A and 4B, the pulsed laser power may be changed according to the irradiation time. The decrease form determination unit 112-1 of the laser power control unit 112 may select the decrease form of the laser power as a linear form or an exponential form. The laser power of the pulsed laser may be set to 100 W to 100 kW, the pulse length thereof may be set to 0.5 μs to 300 μs, and the repetition rate thereof may be set to 1 Hz to 100 Hz.

In the case of the general pulsed laser, the laser energy is constantly and periodically generated with time. That is, when the laser energy is supplied in the pulsed form, the energy is generated in the on and off form. Thus, a case in which the energy is transmitted to the tissue and a case in which the energy is not transmitted to the tissue repeatedly occurs.

When the laser irradiation device 1000 according to the embodiment of the present invention is used, the laser power may be irradiated in a continuous decrease form according to the irradiation time. The decrease form may decrease the laser power (laser pulse energy) linearly according to the irradiation time or may decrease the laser power exponentially according to the irradiation time.

After the decrease form is determined, the decrease rate of the laser power may be determined according to the irradiation time, coagulation thickness in the tissue, and tissue temperature for coagulation induction. Upon linear decrease, when the slope decreases in the exponential form, the magnitude of the exponent may be adjusted.

In order to adjust the pulse energy, the length of t₁ may be adjusted between 0.5 μs to 300 μs, and the length of t₂ may be determined according to the set repetition rate (for example, 10 ms to 1 s).

The temperature in the tissue increases while the laser energy is irradiated during t₁, and the temperature in the tissue decreases due to cooling when the laser energy is not irradiated during t₂.

In the case of the general pulsed laser, the pulse energy is constantly generated, but in the case of the laser irradiation device 1000 according to the embodiment of the present invention, the intensity of the laser power decreases with time. Thus, the total energy amount transmitted to the tissue may be relatively small. Meanwhile, the laser irradiation device 1000 according to the embodiment of the present invention increases initial pulse energy so as to cause photocoagulation during laser irradiation, thereby keeping the total energy amount equal.

Meanwhile, a difference between the pulsed laser and the modulated continuous wave laser is that the continuous wave laser has a very long pulse length (continuous wae: 0.1 ms to 1,000 ms vs. pulsed wave: 0.5 μs to 300 μs), and each pulse energy is relatively very low.

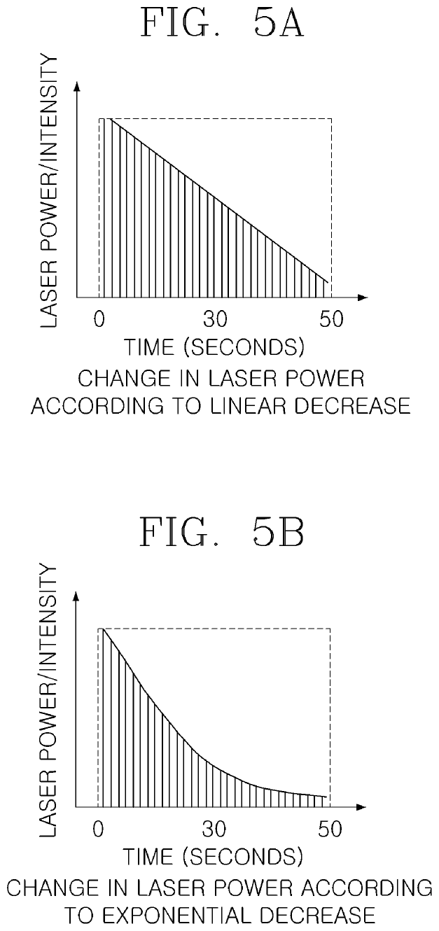

FIGS. 5A and 5B are graphs showing a change in laser power when a laser wavelength is in a quasi-continuous pulsed form, upon laser irradiation through the laser irradiation device 1000 according to the embodiment of the present invention.

As illustrated in FIGS. 5A and 5B, the quasi-continuous pulsed laser power may be changed according to the irradiation time. The decrease form determination unit 112-1 of the laser power control unit 112 may select the decrease form of the laser power as a linear form or an exponential form. The laser power of the pulsed laser may be set to 100 W to 100 kW, the pulse length thereof may be set to 10 ns to 250 ns, and the repetition rate thereof may be set to 5 kHz to 350 kHz.

In the case of the general pulsed laser, the laser energy is constantly and periodically generated with time. That is, when the laser energy is supplied in the pulsed form, the energy is generated in the on and off form. Thus, a case in which the energy is transmitted to the tissue and a case in which the energy is not transmitted to the tissue repeatedly occurs.

When the laser irradiation device 1000 according to the embodiment of the present invention is used, the laser power may be irradiated in a continuous decrease form according to the irradiation time. The decrease form may decrease the laser power (laser pulse energy) linearly according to the irradiation time or may decrease the laser power exponentially according to the irradiation time.

After the decrease form is determined, the decrease rate of the laser power may be determined according to the irradiation time, coagulation thickness in the tissue, and tissue temperature for coagulation induction. Upon linear decrease, when the slope decreases in the exponential form, the magnitude of the exponent may be adjusted.

When the pulse energy is transmitted quasi-continuously, the magnitude of each pulse energy is continuously reduced, such that the temperature in the tissue is not quickly increased, but is constantly maintained.

In the case of the general quasi-continuous pulsed wave laser, short laser pulse energy is constantly and continuously generated, but in the case of the laser irradiation device 1000 according to the embodiment of the present invention, short pulse energy continuously decreases with time. Thus, the total energy amount transmitted to the tissue may be relatively small. Meanwhile, the laser irradiation device 1000 according to the embodiment of the present invention increases initial pulse energy so as to cause photocoagulation during laser irradiation, thereby keeping the total energy amount equal.

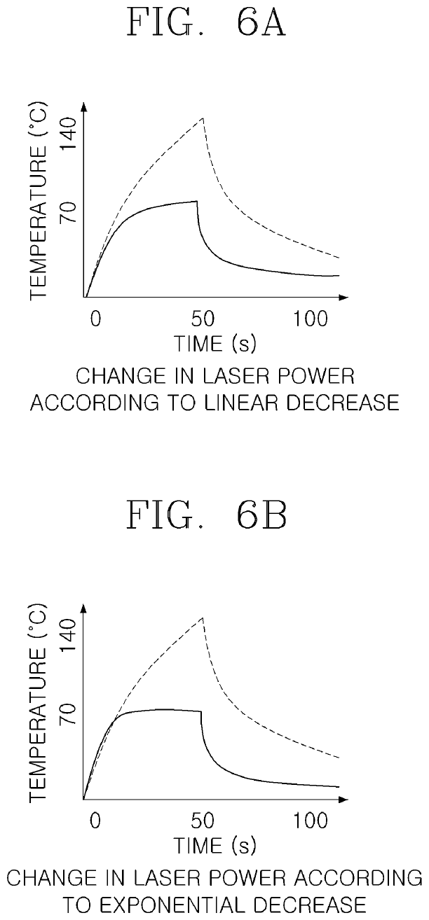

FIGS. 6A and 6B are graphs showing a temperature change in a tissue when a laser wavelength is in a continuous form, upon laser irradiation through the laser irradiation device 1000 according to an embodiment of the present invention;

Unlike the related art in which the temperature in the tissue continuously increases during laser irradiation, the laser irradiation device 1000 according to the embodiment of the present invention maintains the temperature in the tissue at a desired temperature during laser irradiation.

When the temperature in the tissue is increased by using laser energy, the temperature change may be predicted as expressed in Equation 2.

$$( {ρ}_{u} {C}_{a} ) \frac{\partial {T}_{u}}{\partial t} + ∇ ( - {k}_{u} ∇ {T}_{u} ) = {Q}_{laser} \\ {Q}_{laser} = {μ}_{a} I ( r , z , t ) \qquad ( Equation 2 )$$

(ρ=tissue density, c=specific heat of static pressure of tissue, T=tissue temperature, k=tissue heat conductivity, μa=tissue absorption coefficient, I=laser power/energy density in tissue)

For example, when continuous laser is irradiated for about 50 seconds, the temperature in the tissue reaches up to 140° C. so that the tissue surface is carbonized and the tissue is greatly damaged. After the irradiation (after 50 seconds), the temperature in the tissue gradually decreases.

As illustrated in FIGS. 6A and 6B, in the case of using the continuous laser in which the laser power decreases with the irradiation time as in the present invention, the temperature during the irradiation time is maintained at a preset temperature for a predetermined time, thereby preventing carbonization of the tissue surface and easily predicting the degree of tissue damage.

As one example of the method for linearly decreasing the laser power, when a relationship of power=−0.056 (W/s)·x hours (s)+3.5 W, the temperature in the tissue during the irradiation time (50 seconds) can be maintained at 70° C. for 30 seconds (a solid line in FIG. 6A).

As one example of the method for decreasing the laser power exponentially, when a relationship of power=4.97×exp(−t/10)+1.25 W, the temperature in the tissue during the irradiation time (50 seconds) can be maintained at 70° C. for 45 seconds (a solid line in FIG. 6B).

The power relationship may be determined according to the set temperature in the tissue, temperature increase formula, thermal/optical characteristics of the tissue, and the like. Therefore, the temperature in the tissue can be constantly maintained, and the tissue photoreaction may be easily predicted.

Meanwhile, when the laser power is exponentially decreased, the set temperature in the tissue can be maintained longer than 50% as compared with the case of the linear decrease.

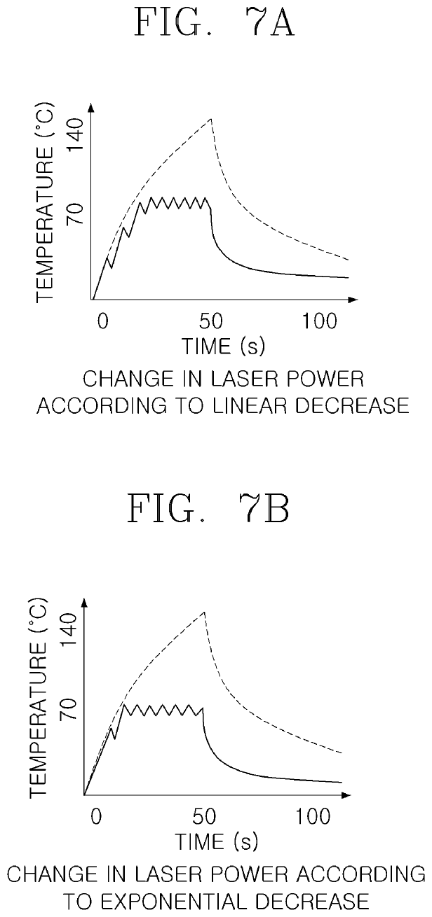

FIGS. 7A and 7B are graphs showing a temperature change in a tissue when a laser wavelength is in a pulsed form, upon laser irradiation through the laser irradiation device 1000 according to an embodiment of the present invention;

When the general pulsed wave laser is used as in the related art, the temperature in the tissue continuously increases during laser irradiation, but the laser irradiation device 1000 according to the embodiment of the present invention maintains the temperature in the tissue at a desired temperature during laser irradiation.

For example, when pulsed wave laser is irradiated for about 50 seconds, the temperature in the tissue reaches up to 140° C. so that the tissue surface is carbonized and the tissue is greatly damaged. After the irradiation (after 50 seconds), the temperature in the tissue gradually decreases.

As illustrated in FIGS. 7A and 7B, in the case of using the pulsed wave laser in which the laser power decreases with the irradiation time as in the present invention, the temperature during the irradiation time is maintained at a preset temperature for a predetermined time, thereby preventing carbonization of the tissue surface and easily predicting the degree of tissue damage.

Unlike the continuous wave laser, each laser energy is transmitted according to pulse irradiation, and the temperature increase gradually increases stepwise. The stepwise gradual increase of the temperature increases according to the laser frequency, and the temperature increase rate is 1 Hz to 100 Hz.

In the case of the method for linearly decreasing the laser power, the temperature is maintained with a width up and down (for example, within ±15%) at a preset temperature (for example, 70° C.), and the average temperature at this time is equal to the preset temperature.

In the case of the method for exponentially decreasing the laser power, the period in which the temperature is maintained at the preset temperature may be made long as compared with the method for linearly decreasing the laser power. In addition, it is possible to quickly reach the set temperature as compared with the linear function (for example, exponential function: 0.7° C./sec vs linear function: 0.4° C./sec). As described above, the decrease form may be determined in consideration of the coagulation range in the tissue.

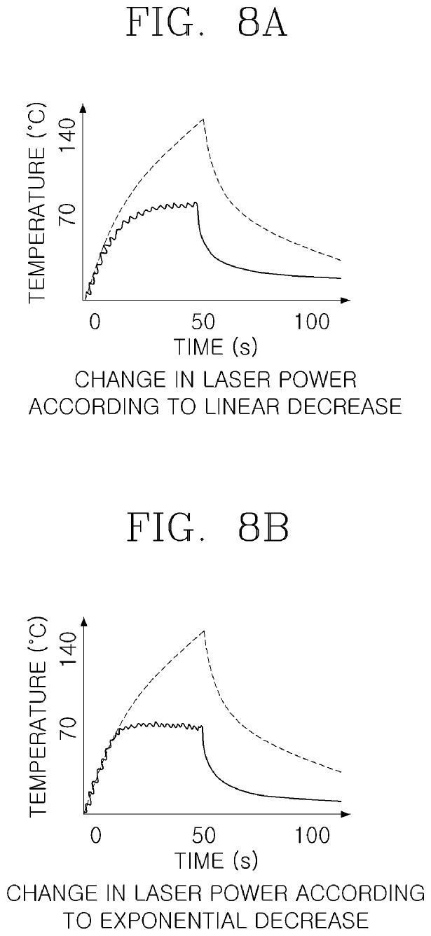

FIGS. 8A and 8B are graphs showing a temperature change in a tissue when a laser wavelength is in a quasi-continuous pulsed form, upon laser irradiation through the laser irradiation device 1000 according to an embodiment of the present invention;

When the general quasi-continuous pulsed wave laser is used as in the related art, the temperature in the tissue continuously increases during laser irradiation, but the laser irradiation device 1000 according to the embodiment of the present invention maintains the temperature in the tissue at a desired temperature during laser irradiation.

For example, when quasi-continuous pulsed wave laser is irradiated for about 50 seconds, the temperature in the tissue reaches up to 140° C. so that the tissue surface is carbonized and the tissue is greatly damaged. After the irradiation (after 50 seconds), the temperature in the tissue gradually decreases.

As illustrated in FIGS. 8A and 8B, in the case of using the pulsed wave laser in which the laser power decreases with the irradiation time as in the present invention, the temperature during the irradiation time is maintained at a preset temperature for a predetermined time, thereby preventing carbonization of the tissue surface and easily predicting the degree of tissue damage.

Unlike the continuous wave laser, each laser energy in the case of the quasi-continuous pulsed wave laser is transmitted according to pulse irradiation, and the temperature increase gradually increases stepwise. Unlike the pulsed wave laser, each laser energy irradiation is very quickly progressed, and the temperature increase is progressed at a higher speed.

The stepwise increase of the temperature increases according to the frequency of the quasi-continuous pulsed laser, and the temperature increase rate is 5 kHz to 35 kHz. A temperature increase in a shorter form is induced as compared with the pulsed laser. (Smaller stepwise increase)

In the case of the method for linearly decreasing the laser power, the temperature is maintained with a width up and down (for example, within ±10%) at a preset temperature (for example, 70° C.), and the average temperature at this time is equal to the preset temperature. As compared with the pulsed laser, the quasi-continuous pulsed laser has small laser pulse energy, and a temperature change rate is small with reference to the set temperature. Thus, precise temperature control is possible.

In the case of the method for exponentially decreasing the laser power, the period in which the temperature is maintained at the preset temperature may be made long as compared with the method for linearly decreasing the laser power. In addition, it is possible to relatively quickly reach the set temperature as compared with the linear function (for example, exponential function: 0.7° C./sec vs linear function: 0.4° C./sec). As described above, the decrease form may be determined in consideration of the coagulation range in the tissue.

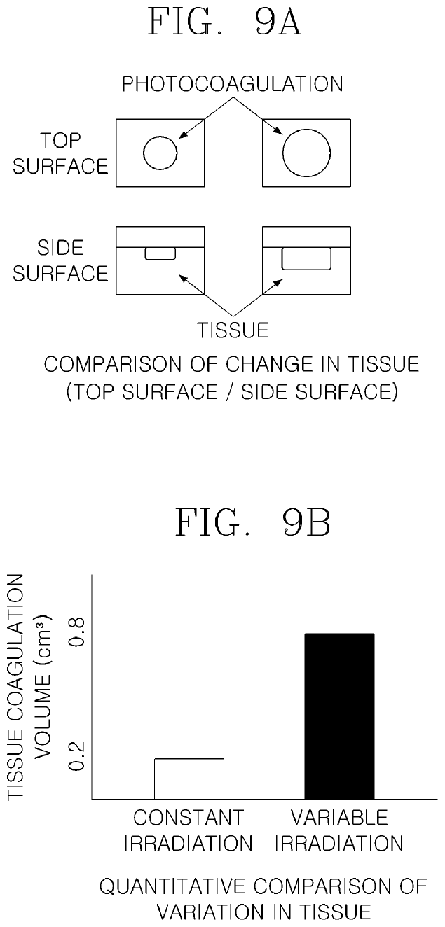

FIGS. 9A and 9B shows comparison of a photocoagulation reaction between a case in which laser is irradiated with constant laser power and a case in which laser is irradiated while reducing laser power with time according to the present invention.

Referring to FIG. 9A, it can be seen that, when the laser power/energy is constantly irradiated, the temperature control is impossible, carbonization occurs on the tissue surface, the formation of the carbonized film makes laser energy absorption and heat transfer become very limited, and the coagulation range in the tissue becomes very small (left side in FIG. 9A).

On the other hand, it can be seen that, when the laser power/energy is irradiated to decrease with time, the temperature control is possible, no carbonization occurs on the tissue surface, laser energy absorption and heat transfer are very efficiently performed, and the coagulation range in the tissue is maximized (right side in FIG. 9A). That is, the coagulated site is formed greater and deeper as compared with the constant irradiation.

For example, when the continuous wave laser having a wavelength of 1,470 nm is irradiated with 2 W to a liver tissue for 50 seconds, the coagulation volume in the tissue is 0.2 cm³ and partial carbonization occurs on the surface. However, in the case of applying the output method in the linear decrease form during irradiation of continuous laser for 50 seconds (power=−0.056 (W/s)·x hours (s)+3.5 W), the tissue surface temperature is maintained at 70° C. for 30 seconds, and the tissue coagulation volume is 0.8 cm³. It can be seen that the tissue coagulation volume during irradiation is greater than four times as compared with the constant irradiation (FIG. 9B).

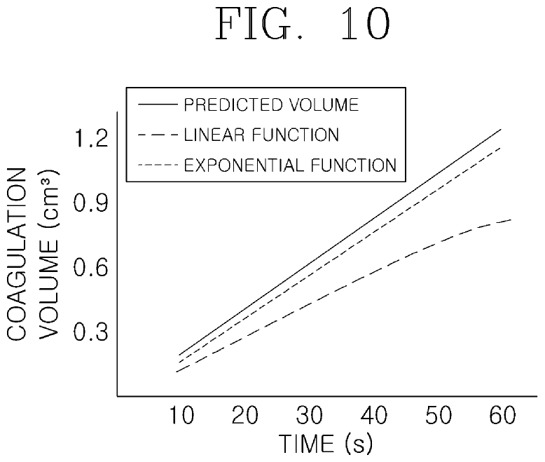

FIG. 10 shows an error of a coagulation volume according to a decrease form, upon laser irradiation through the laser irradiation device 1000 according to the embodiment of the present invention.

Referring to FIG. 10, it can be seen that, when the laser is irradiated while laser power is exponentially decreased, the coagulation volume is close to the expected volume calculated in theory even if the time elapses. It can be seen that, when the laser is irradiated while laser power is linearly decreased, an error is relatively small until a short time (about 30 seconds to 35 seconds), but an error increases as the time becomes long. An appropriate decrease form may be determined in consideration of parameters of at least one of the photocoagulation temperature duration time and the photocoagulation range.

Although preferred embodiments of the present invention have been described for illustrative purposes, those skilled in the art will appreciate that various modifications, additions and substitutions are possible, without departing from the scope and spirit of the invention as disclosed in the accompanying claims. Therefore, the embodiments of the present invention are disclosed only for illustrative purposes and should not be construed as limiting the present invention.

What is claimed is:

1. A laser irradiation device for irradiating laser to a tissue, comprising:
a decrease form determination unit configured to determine a decrease form of laser power according to a laser irradiation time;
a decrease rate determination unit configured to determine a decrease rate of the determined decrease form;
laser light source unit configured to emit laser in the determined decrease form and the determined decrease rate; and
a laser irradiation unit configured to receive laser emitted from the laser light source unit and irradiate the received laser.

2. The laser irradiation device of claim 1, wherein the decrease form is determined in consideration of parameters of at least one of a photocoagulation duration time and a coagulation range in a tissue.

3. The laser irradiation device of claim 2, wherein the decrease form is a linear decrease form.

4. The laser irradiation device of claim 2, wherein the decrease form is an exponential decrease form.

5. The laser irradiation device of claim 1, wherein the decrease rate is determined in consideration of parameters of at least one of an irradiation time, a coagulation thickness in a tissue, and a tissue temperature for coagulation induction.

6. The laser irradiation device of claim 1, wherein, when the laser is in a continuous wave form, a temperature in the tissue is maintained at a specific temperature for a predetermined time, upon laser irradiation through the laser irradiation device.

7. The laser irradiation device of claim 1, wherein, when the laser is in a pulsed or quasi-continuous pulsed form, an average value of temperatures in the tissue is maintained at a specific value for a predetermined time, upon laser irradiation through the laser irradiation device.
